(19) United States
(12) Patent Application Publication
(10) Pub. No.: US 2014/0073804 A1
(43) Pub. Date: Mar. 13, 2014

(54) PROCESS FOR THE PREPARATION OF ZANAMIVIR

(71) Applicants: Ganpat Dan Shimbhu Charan, Gujarat (IN); Ajay Onkarsingh Tehare, Gujarat (IN); Kumar Kemlesh Laxmi Singh, Gujarat (IN)

(72) Inventors: Ganpat Dan Shimbhu Charan, Gujarat (IN); Ajay Onkarsingh Tehare, Gujarat (IN); Kumar Kemlesh Laxmi Singh, Gujarat (IN)

(21) Appl. No.: 14/001,320

(22) PCT Filed: Jun. 13, 2011
(86) PCT No.: PCT/IN2011/000394

§ 371 Date: Nov. 6, 2013

(30) Foreign Application Priority Data
Feb. 24, 2011 (IN) 507/MUM/2011

(51) Int. Cl. C07D 309/28

(52) U.S. Cl. CPC C07D 309/28
USPC 549/424

(57) ABSTRACT

The present invention provides a process for preparing 5-(acetylamino)-4-[(aminoiminomethyl)amino]-2,6-anhydro-3,4,5-trideoxy-D-glycero-D-galacto-non-enonic acid Formula (I), which process comprises reducing compound of Formula (IV) by Lindlar catalyst in presence of hydrogen to obtain compound of Formula (V). reacting compound of Formula (V) with pyrazole-1H-carboxamidine or its suitable salt to obtain compound of Formula (VIII). hydrolyzing the compound of Formula (VIII) to give compound of Formula (I). The present invention also provides compounds of formula (VIII) which may be used in the synthesis of zanamivir. The present invention also provides process for preparing compound of formula (VIII) and process involving the use of Formula (VIII), including in the synthesis of zanamivir.

### FIELD OF THE INVENTION

The present invention relates to synthesis of zanamivir of Formula (I). The invention further relates to a novel intermediate useful in the preparation of compound of Formula (I) and processes for their preparation.

### BACKGROUND OF THE INVENTION

Zanamivir is the first neuraminidase inhibitor to be developed commercially, and it is used in the treatment of and prophylaxis of both Influenza virus A and Influenza virus B. Chemically, zanamivir is 5-(acetylamino)-4-[(aminoiminomethyl)amino]-2,6-anhydro-3,4,5-trideoxy-D-glycero-D-galacto-non-enonic acid (Formula I), and is represented by the following Structure:

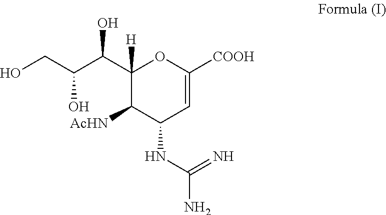 [SMILES: CC(=O)N[C@H]1[C@H]([C@H](O)[C@H](O)CO)OC(C(=O)O)=C[C@@H]1NC(=N)N]

Zanamivir binds to the conserved region of influenza neuraminidase enzyme, which mainly catalyzes the cleavage of terminal sialic acid attached to glycolipids and glycoproteins.

The preparation and use of derivatives and analogs of 2-deoxy-2,3-didehydro-N acetylneuraminic acid, are disclosed in U.S. Pat. No. 5,360,817 which are antiviral agents. Disclosed therein are compounds, whose general formula is,

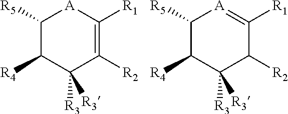

The process for preparation of zanamivir was first described in the U.S. Pat. No. 5,360,817 wherein, selective deacetylation of 5-acetamido-4-acetoxy-6-(1,2,3-triacetoxypropyl)-5,6-dihydro-4H-pyran-2-carboxylate of Formula (II) with boron trifluoride ethearate gives 5-acetamido-4-hydroxy-6-(1,2,3-triacetoxypropyl)-5,6-dihydro-4H-pyran-2-carboxylate of

Formula (III), which on further treatment with trifluoromethanesulfonic anhydride and sodium azide gives 5-acetamido-4-azido-6-(1,2,3-triacetoxypropyl)-5,6-dihydro-4H-pyran-2-carboxylate of Formula (IV). The reduction of intermediate compound of Formula (IV) with hydrogen sulphide in pyridine affords the corresponding 5-acetamido-4-amino-6-(1,2,3-triacetoxypropyl)-5,6-dihydro-4H-pyran-2-carboxylate intermediate of Formula (V), which is finally condensed with S-methylisothiourea in water and saponified through Dowex 50 W in aqueous ammonium hydroxide to yield zanamivir (I).

The problems associated with the disclosed process are that even on passing hydrogen sulphide gas for around 16 hours, there is no complete reduction of the 4-azido intermediate into the 4-amino compound. Also, due to the excessive use of the gas, there is a risk of undesired reduction of the 2,3-double bond along with the 4-azido group. The over-reduction leads to formation of undesired products which need additional purification procedures in order to separate the undesired products. Also all over yield of the reaction was very low.

Another process for preparation of compound of Formula (I) is disclosed in WO94/07885 which involves preparation by treating 5-acetamido-4-amino-6-(1,2,3-trihydroxypropyl)-5,6-dihydro-4H-pyran-2-carboxylic acid of Formula (VI), which is obtained as per WO91/16320, with cyanogen bromide in the presence of sodium acetate to yield 4-cyanoamide derivative (VII) which is further reacted with ammonium formate and ammonia to yield compound of Formula (I).

EP0539204 also discloses the preparation of zanamivir by treating cyanoamide derivative (VII) with an amine derivative or treating 4-amino compound (VI) with a carbamimidic compound.

U.S. Pat. No. 5,495,027 discloses the use of a Lindlar catalyst (lead doped palladium catalyst) for the conversion of azide to amine and the product of reduction is subsequently hydrolysed in aqueous medium to form zanamivir.

EP 662967 discloses the synthesis of zanamivir by reacting the 5-acetamido-4-amino-6-(1,2,3-trihydroxypropyl)-5,6-dihydro-4H-pyran-2-carboxylic acid (VI) with pyrazole-1H-carboxamidine.

PCT publication No. WO 2010061182 describes a process of preparation of zanamivir. The product is prepared by reducing methyl 5-acetamido-4-azido-6-(1,2,3-triacetoxypropyl)-5,6-dihydro-4H-pyran-2-carboxylate of Formula (IV) in the presence of a reducing agent selected from the group consisting of lithium aluminium hydride, sodium borohydride, zinc/ammonium chloride, zinc-ferric chloride and ferric chloride/sodium iodide.

Several of the above processes either have a problem of low yield and purity or are difficult to carry out on a commercial scale and are expensive. Hence there is a high unmet need to develop a process which provides the compound of Formula (I) at low cost and which should be environment friendly, scalable, and industrially applicable. The present invention provides a process which is efficient, cost effective and does not result in impure product, thus making the process amenable for commercial scale use.

### SUMMARY OF THE INVENTION

In one general aspect there is provided a process for the preparation of compound of Formula (I).

The process includes:

(a) reduction of compound of Formula (IV):

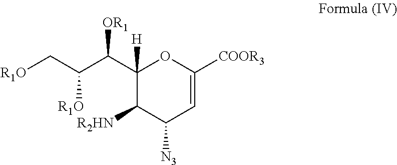

by a Lindlar catalyst in presence of hydrogen in suitable solvents to obtain compound of Formula (V).

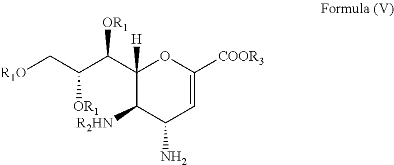

where R₁ is suitable hydroxyl protecting group selected from aralkyl groups such as benzyl, diphenyl methyl or triphenyl methyl group and the like; acyl groups such as acetyl and the like; silicon containing protecting groups such as trimethylsilyl groups or as tetrahydropyran derivatives and the like; R₂ is amino protecting groups selected from aralkyl groups such as benzyl, diphenylmethyl or triphenylmethyl group and the like; acyl groups such as acetyl, N-benzyloxy carbonyl or t-butoxycarbonyl and R₃ is C(1-4) alkyl group.
(b) reacting compound of Formula (V) with pyrazole-1H-carboxamidine or its suitable salt to obtain compound of Formula (VIII).

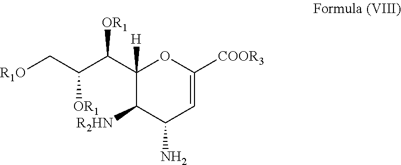

wherein R₁, R₂, R₃ are as defined earlier.
(c) hydrolyzing the compound of Formula (VIII) to give compound of Formula (I).
(d) Optionally, further purifying the compound of Formula (I) by using suitable solvent and by adding suitable base to obtain pure compound of Formula (I).

In one aspect there is provided a process for the preparation of compound of Formula (I). The process includes:

(a) reduction of 5-acetamido-4-azido-6-(1,2,3-triacetoxypropyl)-5,6-dihydro-4H-pyran-2-carboxylate of Formula (IV):

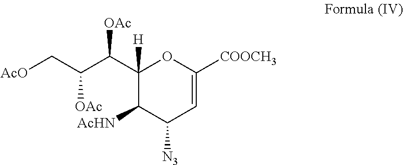 [SMILES: COC(=O)C1=C[C@H](N=[N+]=[N-])[C@@H](NC(C)=O)[C@H]([C@H](OC(C)=O)[C@@H](COC(C)=O)OC(C)=O)O1]

by a Lindlar catalyst in presence of hydrogen in suitable solvents to obtain compound of Formula (V).

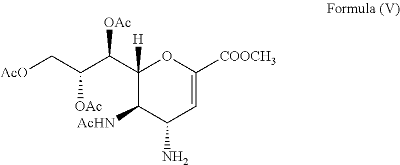 [SMILES: COC(=O)C1=C[C@H](N)[C@@H](NC(C)=O)[C@H]([C@H](OC(C)=O)[C@@H](COC(C)=O)OC(C)=O)O1]

(b) reacting compound of Formula (V) with pyrazole-1H-carboxamidine or its suitable salt to obtain compound of Formula (VIII).

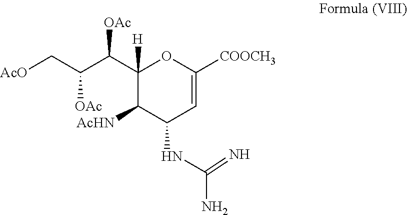 [SMILES: COC(=O)C1=C[C@H](NC(=N)N)[C@@H](NC(C)=O)[C@H]([C@H](OC(C)=O)[C@@H](COC(C)=O)OC(C)=O)O1]

(c) hydrolyzing the compound of Formula (VIII) to give compound of Formula (I).
(d) Optionally, further purifying the compound of Formula (I) by using suitable solvent and by adding suitable base to obtain pure compound of Formula (I).

In another general aspect there is provided compound of Formula (VIII) and their use for the preparation of compound of Formula (I).

The process may further include converting the product so obtained as above, into a finished dosage form.

The details of one or more embodiments of the invention are set forth in the description below. Other features, objects and advantages of the invention will be apparent from the description and claims.

Embodiments of the process may include one or more of the following features. For example, the protection of hydroxyl group R₁ and amino group R₂ of Formula IV may be carried out in the presence of a suitable protecting agent and one or more suitable solvents. The suitable protecting agent may be selected from those disclosed in Text book—Title: ‘Protective Groups in Organic Synthesis’ 3rd Edition, John Wiley & Sons, By-T. W. Grene and Peter G. M. Wuts) which also describes methods for the removal of such groups.

### DETAILED DESCRIPTION OF THE INVENTION

As used herein, the term “reflux temperature” refers to the boiling point of the solvent being used in the corresponding step.

As used herein, the term “THF” refers to tetrahydrofuran, the term “DCM” refers to dichloro methane, the term “TEA” refers to triethyl amine, the term “DMF” refers to dimethyl formamide, the term “DIPE” refers to di-isopropyl ether, the term “MTBE” refers to methyl t-butyl ether, the term “DMSO” refers to dimethyl sulfoxide, the term “DMA” refers to dimethylacetamide, the term “IPA” refers to isopropyl alcohol. As used herein, the term “DBU” refers to 1,8-diazabicyclo [5.4.0] undec-7-ene.

The inventors have developed a process for the preparation of compound of Formula (I). The process includes:

(a) reduction of compound of Formula (IV):

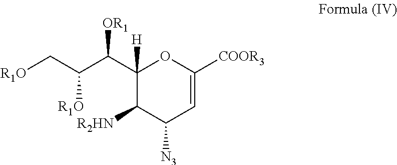

by Lindlar catalyst in presence of hydrogen in suitable solvents to obtain compound of Formula (V).

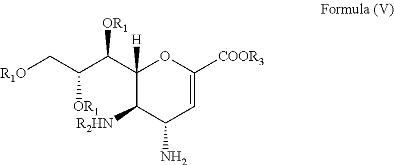

where R₁ is suitable hydroxyl protecting group selected from aralkyl groups such as benzyl, diphenyl methyl or triphenyl methyl group and the like; acyl groups such as acetyl and the like; silicon containing protecting groups such as trimethylsilyl group or as tetrahydropyran derivatives and the like; R₂ is amino protecting groups selected from aralkyl groups such as benzyl, diphenylmethyl or triphenylmethyl group and the like; acyl groups such as acetyl, N-benzyloxy carbonyl or t-butoxycarbonyl and R₃ is C(1-4) alkyl group.
(b) reacting compound of Formula (V) with pyrazole-1H-carboxamidine or its suitable salts to obtain compound of Formula (VIII).

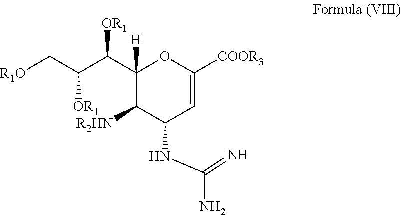

wherein R₁, R₂, R₃ are as defined earlier.
(c) hydrolyzing the compound of Formula (VIII) to give compound of Formula (I).
(d) optionally, purifying the compound of Formula (I) by using suitable solvent and by adding suitable base to obtain pure compound of Formula (I).

In another general aspect there is provided a process of compound of Formula (VIII).

The process includes:
(a) reduction of compound of Formula (IV):

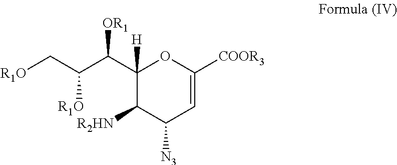

by Lindlar catalyst in presence of hydrogen in suitable solvents to obtain compound of Formula (V).

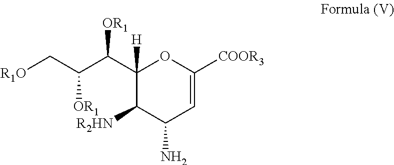

wherein R₁, R₂, R₃ are as defined earlier.
(b) reacting compound of Formula (V) with pyrazole-1H-carboxamidine or its suitable salts to obtain compound of Formula (VIII).

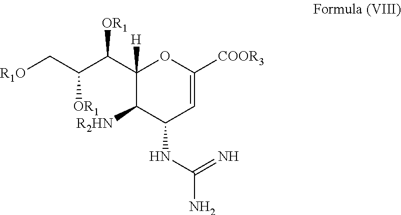

Embodiments of the process may include one or more of the following features. For example, the protection of hydroxyl group R₁ and amino group R₂ of Formula IV may be carried out in the presence of a suitable protecting agent and one or more suitable solvents. The suitable protecting agent may be selected from those disclosed in Text book—Title: ‘Protective Groups in Organic Synthesis’ 3rd Edition, John Wiley & Sons, By-T. W. Grene and Peter G. M. Wuts) which also describes methods for the removal of such groups.

In an embodiment, the term alcohols used anywhere in the specification, unless otherwise specified means suitable (C₁-C₆) linear or branched chain alcohols, more preferably those that are selected from methanol, ethanol, isopropanol, butanol, 1,2-dimethoxy ethanol, 2-methoxy ethanol, 2-ethoxy ethanol, ethylene glycol or their suitable mixtures.

In an embodiment, the term chlorinated solvents used anywhere in the specification, unless otherwise specified would mean chlorine containing solvents, preferably those selected from chloroform, dichloromethane, dichloroethane or their suitable mixtures.

In an embodiment, the term nitriles used anywhere in the specification, unless otherwise specified are selected from acetonitrile and the likes.

In an embodiment, the term aprotic polar solvents used anywhere in the specification, unless otherwise specified may be selected from DMF, DMA, N*methylpyrrolidone or their suitable mixtures.

In an embodiment, the term ethers used anywhere in the specification, unless otherwise specified may be selected from diethyl ether, 1,4-dioxane, dimethoxy ethane, DIPE, MTBE, THF or their suitable mixtures.

In an embodiment, the term esters used anywhere in the specification, unless otherwise specified may be selected from ethyl acetate, isopropyl acetate or their suitable mixtures.
The process is exemplified in greater details below:

### Step-(a)

In general, the reduction of Formula (IV) with Lindlar catalyst in the presence of hydrogen to give compound of Formula (V) may be carried out using suitable solvents. Suitable solvents which can be used at step-(a) may include one or more of alcohols such as methanol, ethanol, isopropanol, butanol, 1,2-dimethoxy ethanol, 2-methoxy ethanol, 2-ethoxy ethanol and ethylene glycol; ethers such as diethyl ether, 1,4-dioxane, dimethoxy ethane, DIPE, MTBE, THF; chlorinated solvents such as chloroform, dichloromethane, dichloroethane; nitriles such acetonitrile; ketones such as acetone, methyl ethyl ketone; aprotic polar solvents such as DMF, DMA, N-methyl pyrrolidone and the like or their suitable mixtures.

Reaction is carried out at temperature 10-100° C., preferably at 15-50° C., more preferably at room temperature.

In an embodiment, the compound of Formula (V) can be isolated or it may be generated in situ and used for next step.

### Step-(b)

The reaction of compound of Formula (V) with pyrazole-1H-carboxamidine or its suitable salt may be carried out using suitable solvents to obtain compound of Formula (VIII).

Suitable solvents which can be used at step-(b) may include one or more of water, alcohols such as methanol, ethanol, isopropanol, butanol, 1,2-dimethoxy ethanol, 2-methoxy ethanol, 2-ethoxy ethanol and ethylene glycol; ethers such as diethyl ether, 1,4-dioxane, dimethoxy ethane, DIPE, MTBE, THF; esters such as ethyl acetate and isopropyl acetate; chlorinated solvents such as chloroform, dichloromethane, dichloroethane; nitriles such acetonitrile; ketones such as acetone, methyl ethyl ketone; aprotic polar solvents such as DMF, DMA, N-methylpyrrolidone and the like or their suitable mixtures.

In an embodiment, the Pyrazole-1H-carboxamidine may first be converted to its suitable acid addition salts such as hydrochloride, hydrobromide, acetate, sulfate and benzene sulfonate, preferably hydrochloride.

In an embodiment, the compound of Formula (VIII) can be isolated or it may be generated in situ and used for the next step.

### Step-(c)

The hydrolysis of Formula (VIII) may be carried out using a suitable base in the presence of suitable solvents to obtain compound of Formula (I).

Suitable solvents which can be used at step-(c) may include one or more of ethers such as diethyl ether, 1,4-dioxane, dimethoxy ethane, DIPE, MTBE, THF; chlorinated solvents such as chloroform, dichloromethane, dichloroethane; nitrites such as acetonitrile; aprotic polar solvents such as DMF, DMA, DMSO; N-methylpyrrolidone, HMPA and the like or their suitable mixtures.

Suitable base(s) used in step (c) may include one or more of DBU; tertiary amines such as triethyl amine, trimethyl amine, triisopropyl amine and diisopropyl ethylamine, preferably triethyl amine; alkali metal alkoxides such as sodium ethoxide, sodium methoxide, potassium t-butoxide, sodium t-butoxide and like, preferably DBU.

The duration of the reaction may vary from 1 to 5 hrs, more specifically 1 to 2 hrs.

### Step-(d)

Purification of the crude compound of Formula (I) by using suitable solvent and by adding suitable base preferably, DBU to obtain pure compound of Formula (I).

Suitable solvents which can be used at step-(d) may include one or more of ethers such as diethyl ether, 1,4-dioxane, dimethoxy ethane, DIPE, MTBE, THF; chlorinated solvents such as chloroform, dichloromethane, dichloroethane; nitriles such as acetonitrile; aprotic polar solvents such as DMF, DMA, DMSO; N-methylpyrrolidone, HMPA and the like or their suitable mixtures.

In a preferred embodiment, the compound of Formula (I), having purity of at least ≧99% is prepared according to the present invention.

In a preferred embodiment there is provided a process for the preparation of compound of Formula (I). The process includes:

(a) reduction of 5-acetamido-4-azido-6-(1,2,3-triacetoxypropyl)-5,6-dihydro-4H-pyran-2-carboxylate of Formula (IV):

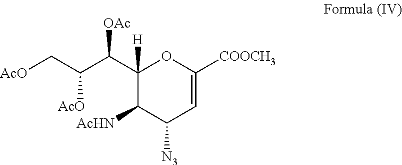 [SMILES: COC(=O)C1=C[C@H](N=[N+]=[N-])[C@@H](NC(C)=O)[C@H]([C@H](OC(C)=O)[C@@H](COC(C)=O)OC(C)=O)O1]

by Lindlar catalyst in presence of hydrogen in suitable solvents to obtain compound of Formula (V).

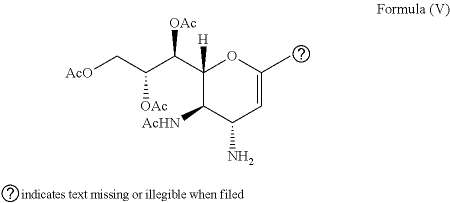 [SMILES: CC(=O)N[C@H]1[C@H]([C@H](OC(C)=O)[C@@H](COC(C)=O)OC(C)=O)OC(C)=C[C@@H]1N]

(b) reacting compound of Formula (V) with pyrazole-1H-carboxamidine or its suitable salts to obtain compound of Formula (VIII).

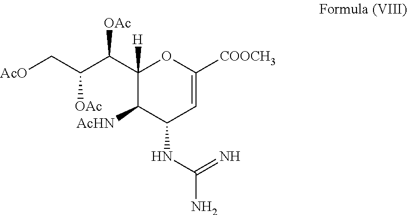 [SMILES: COC(=O)C1=C[C@H](NC(=N)N)[C@@H](NC(C)=O)[C@H]([C@H](OC(C)=O)[C@@H](COC(C)=O)OC(C)=O)O1]

(c) hydrolyzing the compound of Formula (VIII) to give compound of Formula (I).
(d) optionally, purifying the compound of Formula (I) by using suitable solvent and by adding suitable base to obtain pure compound of Formula (I).

In a preferred embodiment there is provided a process for the preparation of compound of Formula (VIII). The process includes:

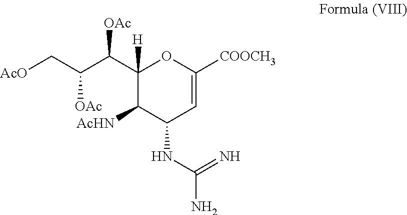 [SMILES: COC(=O)C1=C[C@H](NC(=N)N)[C@@H](NC(C)=O)[C@H]([C@H](OC(C)=O)[C@@H](COC(C)=O)OC(C)=O)O1]

(a) reduction of 5-acetamido-4-azido-6-(1,2,3-triacetoxypropyl)-5,6-dihydro-4H-pyran-2-carboxylate of Formula (IV):

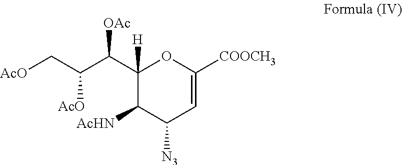 [SMILES: COC(=O)C1=C[C@H](N=[N+]=[N-])[C@@H](NC(C)=O)[C@H]([C@H](OC(C)=O)[C@@H](COC(C)=O)OC(C)=O)O1]

by Lindlar catalyst in presence of hydrogen in a suitable solvents to obtain compound of Formula (V).

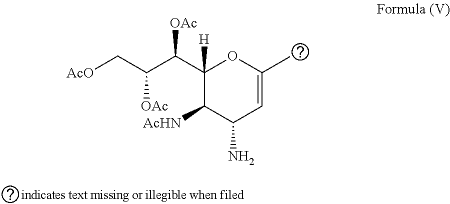 [SMILES: CC(=O)N[C@H]1[C@H]([C@H](OC(C)=O)[C@@H](COC(C)=O)OC(C)=O)OC(C)=C[C@@H]1N]

(b) reacting compound of Formula (V) with pyrazole-1H-carboxamidine or its suitable salts to obtain compound of Formula (VIII).

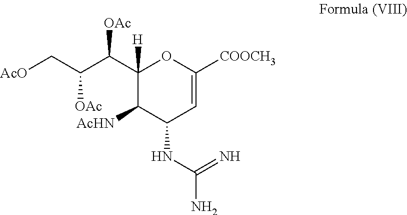 [SMILES: COC(=O)C1=C[C@H](NC(=N)N)[C@@H](NC(C)=O)[C@H]([C@H](OC(C)=O)[C@@H](COC(C)=O)OC(C)=O)O1]

The invention is further exemplified by the following non-limiting examples, which are illustrative representing the preferred modes of carrying out the invention. The invention's scope is not limited to these specific embodiments only but should be read in conjunction with what is disclosed anywhere else in the specification together with those information and knowledge which are within the general understanding of a person skilled in the art. These examples are provided merely as representative embodiments and should not be construed to limit the scope of the invention in any way.

### Example-1

### Process for the preparation of 5-acetamido-4-amino-6-(1,2,3-triacetoxypropyl)-5,6-dihydro-4H-pyran-2-carboxylate (Formula V)

In a suitable hydrogenation vessel, 5-acetamido-4-azido-6-(1,2,3-triacetoxypropyl)-5,6-dihydro-4H-pyran-2-carboxylate (15 gm), methanol were charged under nitrogen atmosphere. The reaction mixture was stirred to get clear solution. Subsequently, 3 gm Lindlar catalyst was added and applied 10-15 PSI hydrogenation pressure and maintained the pressure for 4 hrs. The reaction mass was filtered through Hyflo and wash the hyflobed with methanol. The solvent was completely distilled out under reduced pressure at 50° C. Thick solid material was obtained.

Yield=14.14 g, HPLC purity=91%.

### Example-2

### Process for the preparation of 5-acetamido-4-amino-6-(1,2,3-triacetoxypropyl)-5,6-dihydro-4H-pyran-2-carboxylate (Formula V)

In a suitable hydrogenation vessel, 5-acetamido-4-azido-6-(1,2,3-triacetoxypropyl)-5,6-dihydro-4H-pyran-2-carboxylate (15 gm), methanol and water were charged under nitrogen atmosphere. The reaction mixture was stirred to get clear solution. Subsequently, 3 gm Lindlar catalyst was added and applied 10-15 PSI hydrogenation pressure and maintained the pressure for 4 hrs. The reaction mass was filtered through hyflo and wash the hyflobed with water. The solvent was completely distilled out under reduced pressure at 50° C. Thick solid material was obtained.

Yield=14.14 g, HPLC purity=92%

### Example-3

### Process for the preparation of 5-acetamido-4-guanidino-6-(1,2,3-triacetoxypropyl)-5,6-dihydro-4H-pyran-2-carboxylate (Formula VIII)

In a suitable vessel, 5-acetamido-4-amino-6-(1,2,3-triacetoxypropyl)-5,6-dihydro-′4H-pyran-2-carboxylate (14.14 gm), water, imidazole (6.7 gm) and pyrazole carboxamidine hydrochloride (6.72 gm) were charged. The reaction mixture was stirred to get clear solution. The temperature was raised up to 40 to 45° C. and subsequently, the reaction mixture was maintained at room temperature for 16-18 hrs. The conversion of 5-acetamido-4-amino-6-(1,2,3-triacetoxypropyl)-5,6-dihydro-4H-pyran-2-carboxylate to 5-acetamido-4-guanidino-6-(1,2,3-triacetoxypropyl)-5,6-dihydro-4H-pyran-2-carboxylate was checked by HPLC.

### Example-4

### Process for the preparation of 5-acetamido-4-guanidino-6-(1,2,3-triacetoxypropyl)-6-dihydro-4H-pyran-2-carboxylate (Formula VIII)

In a suitable vessel, 5-acetamido-4-amino-6-(1,2,3-triacetoxypropyl)-5,6-dihydro-′4H-pyran-2-carboxylate (14.14 gm), methanol (45 ml), imidazole (6.7 gm) and pyrazole carboxamidine hydrochloride (6.72 gm) were charged. The reaction mixture was stirred to get clear solution. The temperature was raised up to 40 to 45° C. and subsequently, the reaction mixture was maintained at 40-45° C. for 16-18 hrs. The conversion of 5-acetamido-4-amino-6-(1,2,3-triacetoxypropyl)-5,6-dihydro-4H-pyran-2-carboxylate to 5-acetamido-4-guanidino-6-(1,2,3-triacetoxypropyl)-5,6-dihydro-4H-pyran-2-carboxylate was checked by HPLC.

### Example-5

### Process for the preparation of 5-(acetylamino)-4-[(aminoiminomethyl)amino]-2,6-anhydro-3,4,5-trideoxy-D-glycero-D-galacto-non-enonic acid (Formula I)

Charged TEA (22.48 gm) in a vessel containing 5-acetamido-4-guanidino-6-(1,2,3-triacetoxypropyl)-5,6-dihydro-4H-pyran-2-carboxylate (as prepared in example-2) slowly at temp 5-10° C. Subsequently, the temperature was raised up to RT and maintained for 30 min. The reaction mass was washed with MDC and subsequently, acetone was added. The product thus obtained was isolated. The compound was filtered, washed with acetone and dried under reduced pressure at 50° C.

The yield was 5.5 gm (50%). HPLC Purity—99.0%

### Example-6

### Process for the preparation of 5-(acetylamino)-4-[(aminoiminomethyl)amino]-2,6-anhydro-3,4,5-trideoxy-P-glycero-D-galacto-non-enonic acid (Formula I)

Charged DBU (22.48 gm) in a vessel containing 5-acetamido-4-guanidino-6-(1,2,3-triacetoxypropyl)-5,6-dihydro-4H-pyran-2-carboxylate (as prepared in example-2) slowly at temp 5-10° C. Subsequently, the temperature was raised up to RT and maintained for 30 min. The reaction mass was washed with MDC and subsequently, acetone was added. The product thus obtained was isolated. The compound was filtered, washed with acetone and dried under reduced pressure at 50° C.

The yield was 6.7 g (61.3%). HPLC Purity—99.5%

### Example-7

### Purification of 5-(acetylamino)-4-[(aminoiminomethyl)amino]-2,6-anhydro-3,4,5-trideoxy-D-glycero-D-galacto-non-enonic acid (Formula I)

In a suitable vessel, 5-(acetylamino)-4-[(aminoiminomethyl)amino]-2,6-anhydro-3,4,5-trideoxy-D-glycero-D-galacto-non-enonic acid (5 gm), water and TEA (0.5 ml) were charged. The temperature was raised up to 50 to 55° C. to get clear solution. Subsequently, the reaction mass was treated with activated carbon and filtered at same temperature and acetone was added. The product thus obtained was isolated at temperature at about 40-45° C. The compound was filtered, washed with acetone and dried under reduced pressure at 50° C.

The yield was 5.0 g (83%). HPLC Purity—99.8%.

### Example-8

### Purification of 5-(acetylamino)-4-[(aminoiminomethyl)amino]-2,6-anhydro-3,4,5-trideoxy-P-glycero-D-galacto-non-enonic acid (Formula I)

In a suitable vessel, 5-(acetylamino)-4-[(aminoiminomethyl)amino]-2,6-anhydro-3,4,5-trideoxy-D-glycero-D-galacto-non-enonic acid (5 gm), water and DBU (0.5 ml) were charged. The temperature was raised up to 50 to 55° C. to get clear solution. Subsequently, the reaction mass was treated with activated carbon and filtered at same temperature and acetone was added. The product thus obtained was isolated at temperature at about 40-45° C. The compound was filtered, washed with acetone and dried under reduced pressure at 50° C.

The yield was 5.3 g (88%). HPLC Purity—99.96%.

1. A process for the preparation of zanamivir of Formula (I) comprising, 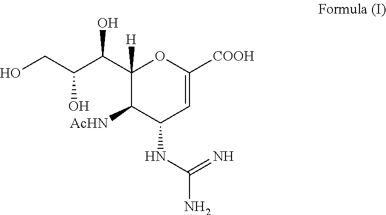 [SMILES: CC(=O)N[C@H]1[C@H]([C@H](O)[C@H](O)CO)OC(C(=O)O)=C[C@@H]1NC(=N)N]
(a) reducing a compound of Formula (IV) 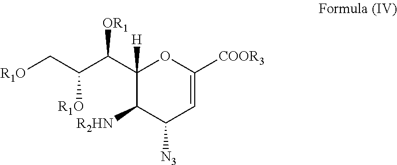
by Lindlar catalyst in presence of hydrogen in a suitable solvent to obtain a compound of Formula (V) 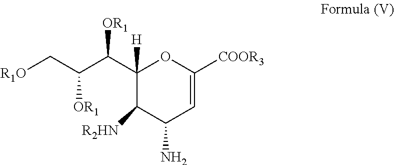
where R₁ represents hydroxyl protecting group selected from aralkyl groups, acyl groups and silicon protecting groups, R₂ represents suitable amino protecting groups selected from aralkyl and acyl groups and R₃ is C₍₁₋₄₎ alkyl group;
(b) reacting the compound of Formula (V) with pyrazole-1H-carboxamidine or a suitable salt thereof in a suitable solvent to obtain a compound of Formula (VIII) 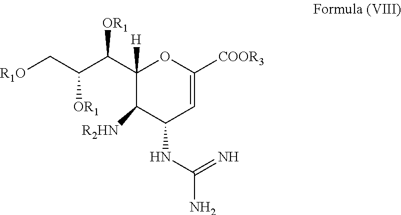
wherein R₁, R₂, and R₃ are as defined earlier;
(c) hydrolyzing the compound of Formula (VIII) in a suitable base in a suitable solvent to obtain a compound of Formula (I) and
(d) optionally, purifying the compound of Formula (I) by using a suitable solvent and by adding a suitable base to obtain a pure compound of Formula (I).

2. The process for the preparation of compound of Formula (I) as claimed in claim 1, wherein the suitable solvent of step (a) is selected from (C₁-C₆) alcohols, ethers, chlorinated solvents, nitriles, ketones, and aprotic polar solvents or a mixture thereof.

3. The process for the preparation of compound of Formula (I) as claimed in claim 1, wherein the suitable salt of an acid of step (b) is selected from hydrochloride, hydrobromide, acetate, sulfate and benzene sulfonate.

4. The process as claimed in claim 3, wherein the salt is hydrochloride salt.

5. The process for the preparation of compound of Formula (I) as claimed in claim 1, wherein the suitable solvent of step (b) is selected from water, (C₁-C₆) alcohols, ethers, esters, chlorinated solvents, nitriles, ketones, and aprotic polar solvents or a mixture thereof.

6. The process for the preparation of compound of Formula (I) as claimed in claim 1, wherein the suitable solvent of step (c) is selected from ethers, chlorinated solvents, nitriles, and aprotic polar solvents or a mixture thereof.

7. The process for the preparation, of compound of Formula (I) as claimed in claim 1, wherein the suitable base of step (c) is selected from one or more of DBU, tertiary amines, and alkali metal alkoxides.

8. The process as claimed in claim 7, wherein the base is DBU.

9. The process as claimed in claim 1, wherein the suitable solvent of step (d) is selected from ethers, chlorinated solvents, nitriles, and aprotic polar solvents or a mixture thereof.

10. The process for the preparation of compound of Formula (I) as claimed in claim 1, wherein the aralkyl groups of step (a) are selected from benzyl, diphenyl methyl and triphenyl methyl group.

11. The process for the preparation of compound of Formula (I) as claimed in claim 1, wherein the acyl group of step (a) is selected from acetyl.

12. The process for the preparation of compound of Formula (I) as claimed in claim 1, wherein the silicon protecting groups of step (a) are selected from trimethylsilyl group and tetrahydropyran derivatives.

13. The process for the preparation of compound of Formula (I) as claimed in claim 1, wherein the amino aralkyl protecting groups of step (a) are selected from benzyl, diphenyl methyl and triphenyl methyl group.

14. The process for the preparation of compound of Formula (I) as claimed in claim 1, wherein the amino acyl protecting groups of step (a) are selected from acetyl, N-benzyloxy carbonyl and t-butoxycarbonyl.

15. An intermediate of formula (VIII) 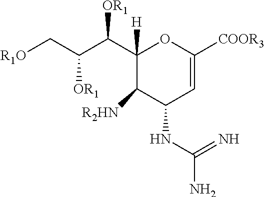
where R₁ represents a hydroxyl protecting group selected from aralkyl groups, acyl groups and silicon protecting groups, R₂ represents an amino protecting group selected from aralkyl group and acyl group and R₃ represents a C₍₁₋₄₎ alkyl group.

16. A process for the preparation of compound of Formula (VIII) as claimed in claim 15, comprising the steps of, 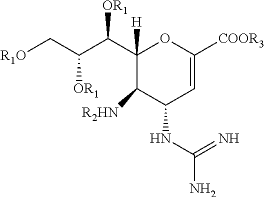
where R₁ is hydroxyl protecting group selected from aralkyl groups, acyl groups and silicon protecting groups, R₂ is amino protecting groups selected from aralkyl group and acyl group and R₃ is C(1-4) alkyl group
(a) reducing a compound of Formula (IV): 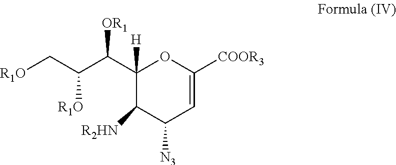
by Lindlar catalyst in presence of hydrogen in a suitable solvent to obtain a compound of Formula (V) 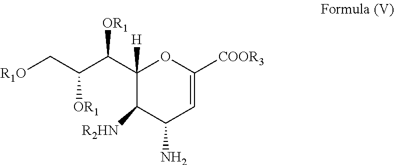
wherein R₁, R₂, and R₁ are as defined above
(b) reacting the compound of Formula (V) with pyrazole-1H-carboxamidine or a suitable salt thereof in a suitable solvent to obtain a compound of Formula (VIII) 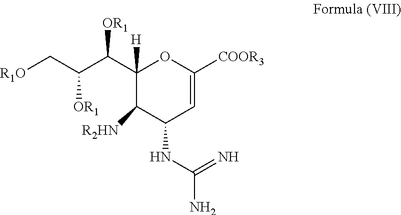
wherein R₁, R₂, and R₃ are as defined in claim 15.

17. The process for the preparation of compound of Formula (VIII) as claimed in claim 16, wherein the suitable solvent of step (a) is selected from (C₁-C₆) alcohols, ethers, chlorinated solvents, nitrites, ketones, and aprotic polar solvents or a mixture thereof.

18. The process for the preparation of compound of Formula III as claimed in claim 16, wherein the aralkyl groups are selected from benzyl, diphenyl methyl and triphenyl methyl group.

19. The process for the preparation of compound of Formula III as claimed in claim 16, wherein the acyl group is selected from acetyl.

20. The process for the preparation of compound of Formula III as claimed in claim 16, wherein the silicon protecting groups is selected from trimethylsilyl group and tetrahydropyran derivatives.

21. The process for the preparation of compound of Formula III as claimed in claim 16, wherein the amino aralkyl protecting groups are selected from benzyl, diphenyl methyl and triphenyl methyl group.

22. The process for the preparation of compound of Formula III as claimed in claim 16, wherein the amino acyl protecting groups is selected from acetyl, N-benzyloxy carbonyl and t-butoxycarbonyl.

23. The process for the preparation of compound of Formula (VIII) as claimed in claim 16, wherein the suitable salt of step (b) is selected from hydrochloride, hydrobromide, acetate, sulfate and benzene sulfonate.

24. The process as claimed in claim 23, wherein the salt is the hydrochloride salt.

25. The process for the preparation of compound of formula (VIII) as claimed in claim 16, wherein the suitable solvent of step (b) is selected from water, (C₁-C₆) alcohols, ethers, esters, chlorinated solvents, nitriles, ketones, and aprotic polar solvents or a mixture thereof.

26. The process for the preparation zanamivir of Formula (I) as claimed in claim 1 comprising
(a) reducing 5-acetamido-4-azido-6-(1,2,3-triacetoxypropyl)-5,6-dihydro-4H-pyran-2-carboxylate of Formula (IV): 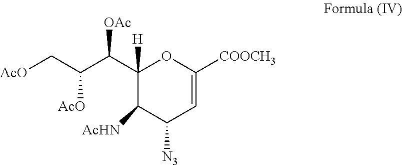 [SMILES: COC(=O)C1=C[C@H](N=[N+]=[N-])[C@@H](NC(C)=O)[C@H]([C@H](OC(C)=O)[C@@H](COC(C)=O)OC(C)=O)O1]
by Lindlar catalyst in presence of hydrogen in a suitable solvent to obtain the compound of Formula (V), 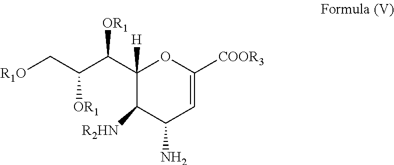
(b) reacting the compound of Formula (V) with pyrazole-1H-carboxamidine or a suitable salt thereof in a suitable solvent to obtain compound of Formula (VIII), 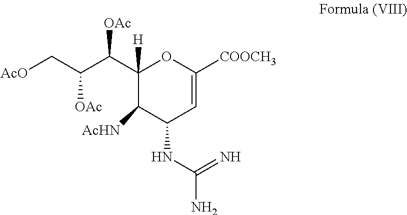 [SMILES: COC(=O)C1=C[C@H](NC(=N)N)[C@@H](NC(C)=O)[C@H]([C@H](OC(C)=O)[C@@H](COC(C)=O)OC(C)=O)O1]
(c) hydrolyzing the compound of Formula (VIII) in a suitable solvent to obtain the compound of Formula (I), and
(d) optionally, purifying the compound of Formula (I) by using suitable solvent and by adding suitable base to obtain a pure compound of Formula (I).

27. The process for the preparation of compound of Formula (I) as claimed in claim 26, wherein the suitable solvent of step (a) is selected from (C₁-C₆) alcohols, ethers, chlorinated solvents, nitriles, ketones, and aprotic polar solvents or a mixture thereof.

28. The process for the preparation of compound of Formula (I) as claimed in claim 26, wherein the suitable salt of an acid of step (b) is selected from hydrochloride, hydrobromide, acetate, sulfate and benzene sulfonate.

29. The process as claimed in claim 28, wherein the salt is hydrochloride salt.

30. The process for the preparation of compound of Formula (I) as claimed in claim 26, wherein the suitable solvent of step (b) is selected from water, (C₁-C₆) alcohols, ethers, esters, chlorinated solvents, nitrites, ketones, and aprotic polar solvents or a mixture thereof.

31. The process for the preparation of compound of Formula (I) as claimed in claim 26, wherein the suitable solvent of step (c) is selected from ethers, chlorinated solvents, nitrites, and aprotic polar solvents or a mixture thereof.

32. The process for the preparation of compound of Formula (I) as claimed in claim 26, wherein the suitable base of step (d) is selected from one or more of DBU, tertiary amines, and alkali metal alkoxides.

33. The process as claimed in claim 32, wherein the base is DBU.

34. The process as claimed in claim 26, wherein the suitable solvent of step (d) is selected from ethers, chlorinated solvents, nitrites, and aprotic polar solvents or a mixture thereof.

35. An intermediate of formula (VIII)

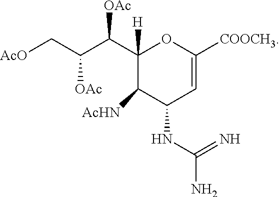 [SMILES: COC(=O)C1=C[C@H](NC(=N)N)[C@@H](NC(C)=O)[C@H]([C@H](OC(C)=O)[C@@H](COC(C)=O)OC(C)=O)O1]

36. A process for the preparation of compound of Formula (VIII) as claimed in claim 35, comprising, 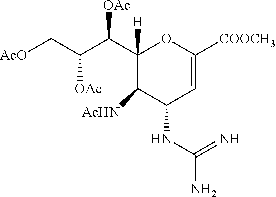 [SMILES: COC(=O)C1=C[C@H](NC(=N)N)[C@@H](NC(C)=O)[C@H]([C@H](OC(C)=O)[C@@H](COC(C)=O)OC(C)=O)O1]
(a) reducing 5-acetamido-4-azido-6-(1,2,3-triacetoxypropyl)-5,6-dihydro4H-pyran-2-carboxylate of Formula (IV) 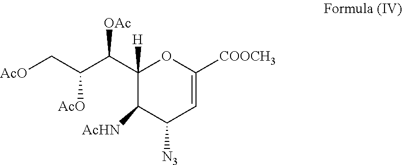 [SMILES: COC(=O)C1=C[C@H](N=[N+]=[N-])[C@@H](NC(C)=O)[C@H]([C@H](OC(C)=O)[C@@H](COC(C)=O)OC(C)=O)O1]
by Lindlar catalyst in presence of hydrogen in a suitable solvent to obtain compound of Formula (V) 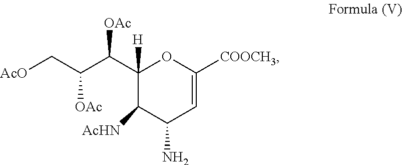 [SMILES: COC(=O)C1=C[C@H](N)[C@@H](NC(C)=O)[C@H]([C@H](OC(C)=O)[C@@H](COC(C)=O)OC(C)=O)O1]
and
(b) reacting the compound of Formula (V) with pyrazole-1H-carboxamidine or a suitable salt thereof in a suitable solvent to obtain compound of Formula (VIII)

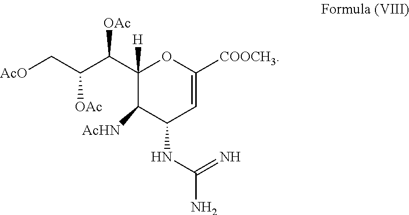 [SMILES: COC(=O)C1=C[C@H](NC(=N)N)[C@@H](NC(C)=O)[C@H]([C@H](OC(C)=O)[C@@H](COC(C)=O)OC(C)=O)O1]

37. The process for the preparation of compound of Formula (VIII) as claimed in claim 36, wherein the suitable solvent of step (a) is selected from (C₁-C₆) alcohols, ethers, chlorinated solvents, nitriles, ketones, and aprotic polar solvents or a mixture thereof.

38. The process for the preparation of compound of Formula (VIII) as claimed in claim 36, wherein the suitable salt of an acid of step (b) is selected from hydrochloride, hydrobromide, acetate, sulfate and benzene sulfonate.

39. The process as claimed in claim 38, wherein the salt is the hydrochloride salt.

40. The process for the preparation of compound of formula (VIII) as claimed in claim 36, wherein the suitable solvent of step (b) is selected from water, (C₁-C₆) alcohols, ethers, esters, chlorinated solvents, nitriles, ketones, and aprotic polar solvents or a mixture thereof.

41. The process as claimed in claim 2, wherein the alcohols are selected from methanol, ethanol, isopropanol, butanol, 1,2-dimethoxy ethanol, 2-methoxy ethanol, 2-ethoxy ethanol, and ethylene glycol or a mixture thereof.

42. The process as claimed in claim 2, wherein the chlorinated solvents are selected from chloroform, dichloromethane, and dichloroethane or a mixture thereof.

43. The process as claimed in claim 2, wherein the nitrile is acetonitrile.

44. The process as claimed in claim 2, wherein the aprotic polar solvents are selected from DMF, DMA, and N-methylpyrrolidone or a mixture thereof.

45. The process as claimed in claim 2, wherein the ethers are selected from diethyl ether, 1,4-dioxane, dimethoxy ethane, DIPE, MTBE, and THF or a mixture thereof.

46. The process as claimed in claim 5, wherein the esters are selected from ethyl acetate, and isopropyl acetate or a mixture thereof.

47. The zanamivir prepared by the process as claimed in claim 1, having purity of at least ≧99%.

48. Zanamivir as claimed in claim 47 having a purity of at least 99.96%.
